(19) United States
(12) Patent Application Publication
(10) Pub. No.: US 2005/0004415 A1
(43) Pub. Date: Jan. 6, 2005

(54) Ion exchange methods of treating a Fischer-Tropsch derived hydrocarbon stream

(75) Inventors: Lucy M. Bull, Pinole, CA (US); Donald L. Kuehne, Hercules, CA (US)

Correspondence Address: BURNS DOANE SWECKER & MATHIS L L P, POST OFFICE BOX 1404, ALEXANDRIA, VA, 22313-1404, US

(73) Assignee: Chevron U.S.A. Inc.

(21) Appl. No.: 10/613,421

(22) Filed: Jul. 2, 2003

(51) Int. Cl. C07C 7/12

(52) U.S. Cl. 585/820

(57) ABSTRACT

Novel methods of treating a Fischer-Tropsch derived hydrocarbon stream with an ion exchange medium are disclosed. Such methods are capable of removing contamination from a Fischer-Tropsch derived hydrocarbon stream such that plugging of the catalyst beds of a subsequent hydroprocessing step is substantially avoided.

### REFERENCE TO RELATED APPLICATIONS

The present application hereby incorporates by reference in its entirety U.S. patent application Ser. No. ______, entitled “Distillation of a Fischer-Tropsch Hydrocarbon Stream Prior to Hydroprocessing, by Richard O. Moore, Jr., Donald L. Kuehne, and Richard E. Hoffer; U.S. patent application Ser. No. ______, entitled “Acid Treatment of a Fischer-Tropsch Hydrocarbon Stream,” by Lucy M. Bull, William Schinski, Donald L. Kuehne, Rudi Heydenrich, and Richard O. Moore, Jr.; and U.S. patent application Ser. No. ______, entitled “Catalytic Filtering of a Fischer-Tropsch Derived Hydrocarbon Stream,” by Jerome F. Mayer, Andrew Rainis, and Richard O. Moore, Jr.

### BACKGROUND OF THE INVENTION

1. Field of the Invention

The present invention relates in general to the processing of products from a Fischer-Tropsch synthesis reaction. More specifically, embodiments of the present invention are directed toward ion exchange methods of removing contamination from a Fischer-Tropsch derived hydrocarbon stream prior to passing that hydrocarbon stream on to additional processing.

2. State of the Art

The majority of the fuel used today is derived from crude oil, and crude oil is in limited supply. However, there is an alternative feedstock from which hydrocarbon fuels, lubricating oils, chemicals, and chemical feedstocks may be produced; this feedstock is natural gas. One method of utilizing natural gas to produce fuels and the like involves first converting the natural gas into an “intermediate” known as syngas (also known as synthesis gas), a mixture of carbon monoxide (CO) and hydrogen (H₂), and then converting that syngas into the desired liquid fuels using a process known as a Fischer-Tropsch (FT) synthesis. A Fischer-Tropsch synthesis is an example of a so-called gas-to-liquids (GTL) process since natural gas is converted into a liquid fuel. Typically, Fischer-Tropsch syntheses are carried out in slurry bed or fluid bed reactors, and the hydrocarbon products have a broad spectrum of molecular weights ranging from methane (C₁) to wax (C₂₀₊).

The Fischer-Tropsch products in general, and the wax in particular, may then be converted to products including chemical intermediates and chemical feedstocks, naphtha, jet fuel, diesel fuel, and lubricant oil basestocks. For example, the hydroprocessing of Fischer-Tropsch products may be carried out in a trickle flow, fixed catalyst bed reactor wherein hydrogen (H₂), or a hydrogen enriched gas, and the Fischer-Tropsch derived hydrocarbon stream comprise the feed to the hydroprocessing reactor. The hydroprocessing step is then accomplished by passing the Fischer-Tropsch derived hydrocarbon stream through one or more catalyst beds within the hydroprocessing reactor, along with a stream of the hydrogen enriched gas.

In some cases, the feeds to be hydroprocessed contain contaminants that originate from upstream processing. These contaminants may take either a soluble or particulate form, and include catalyst fines, catalyst support material and the like, and rust and scale from upstream processing equipment. Fischer-Tropsch wax and heavy products, especially from slurry and fluid bed processes, may contain particulate contaminants (such as catalyst fines) that are not adequately removed by filters provided for that purpose. The removal of those particulates prior to hydroprocessing may be complicated by the potentially high viscosities and temperatures of the wax stream leaving the Fischer-Tropsch reactor.

The typical catalyst used in a hydroprocessing reactor demonstrates a finite cycle time; that is to say, a limited time (or amount) of usefulness before it has to be replaced with a new catalyst charge. The duration of this cycle time usually ranges from about six months to four years or more. It will be apparent to one skilled in the art that the longer the cycle time of a hydroprocessing catalyst, the better the economics of the plant.

Soluble and/or particulate contaminants can create serious problems if they are introduced into the hydroprocessing reactor with the feed. The soluble contaminants pose a problem when, under certain conditions of hydroprocessing, they precipitate out of solution to become particulates. The contamination can cause partial or even complete plugging of the flow-paths through the catalyst beds as the contamination accumulates on the surfaces and interstices of the catalyst. In effect, the catalyst pellets filter out particulate contamination from the feed. In addition to trapping debris that is entrained in the feed, the catalyst beds may also trap reaction by-products from the hydroprocessing reaction itself, an example of such a reaction by-product being coke. Plugging can lead to an impairment of the flow of material through the catalyst bed(s), and a subsequent buildup in the hydraulic pressure-drop across the reactor (meaning the pressure differential between the ends of the reactor where the entry and exit ports are located, respectively). Such an increase in pressure-drop may threaten the mechanical integrity of the hydroprocessing reactor internals.

There are at least two potentially undesirable consequences of catalyst bed plugging. One is a decrease in reactor throughput. A more serious consequence is that a complete shut down of the reactor may be required to replace part or all of the catalyst charge. Either of these consequences can have a negative effect on operating plant economics.

Prior art attempts to manage the problem of catalyst bed plugging in hydroprocessing reactors have been directed toward eliminating at least a portion of the particulate contamination in the feed by filtering the feed prior to its introduction to the hydroprocessing reactor. Such conventional filtration methods are usually capable of removing particulates larger than about 1 microns in diameter. Other prior art methods have been directed toward either controlling the rate of coking on the hydroprocessing catalyst, selecting a feed that is not likely to produce coke, or judiciously choosing the hydroprocessing conditions (conditions such as hydrogen partial pressure, reactor temperature, and catalyst type) that affect coke formation.

The present inventors have found, however, that the above-mentioned open art methods are not effective at removing very small sized particle (or soluble) contaminants, fouling agents, and/or plugging-precursors (hereinafter referred to as “contamination”) from the feedstream to a hydroprocessing reactor when that feedstream comprises a Fischer-Tropsch derived hydrocarbon stream. This is particularly true when the Fischer-Tropsch derived hydrocarbon stream is a wax produced by a slurry bed or fluid bed process. Typical open art methods have therefore not been found to be effective at avoiding the pressure-drop buildup in a hydroprocessing, hydroisomerization, or hydrotreating reactor when that buildup is caused either by particulate contamination, or by soluble contamination that precipitates out of solution.

The apparent failure of typical open art methods has been attributed to either the presence in the hydroprocessing reactor feed of finely divided, solid particulates with diameters of less than about one micron, and/or to a soluble contaminant, possibly having a metallic component, with the ability to precipitate out of solution adjacent to or within the hydroprocessing reactor catalyst beds. What is needed is a method of removing particulates, contaminants, soluble contamination, fouling agents, and plugging precursors from the feedstream to a hydroprocessing reactor such that pressure drop buildup within the hydroprocessing reactor is substantially avoided.

### SUMMARY OF THE INVENTION

A Fischer-Tropsch synthesis is an example of a so-called gas-to-liquids (GTL) process, where natural gas is first converted into syngas (a mixture substantially comprising carbon monoxide and hydrogen), and the syngas is then converted into the desired liquid fuels. Typically, Fischer-Tropsch syntheses are carried out in slurry bed or fluid bed reactors, and the hydrocarbon products have a broad spectrum of molecular weights ranging from methane (C₁) to wax (C₂₀₊). The Fischer-Tropsch products in general, and the wax in particular, may then be hydroprocessed to form products in the distillate fuel and lubricating oil range. According to embodiments of the present invention, hydroprocessing may be conducted in either an upflow or downflow mode. The present process is particularly applicable to operation in the downflow mode.

In some cases, the feeds to be hydroprocessed contain contamination that originates from upstream processing. This contamination may include catalyst fines, catalyst support material and the like, and rust and scale from upstream processing equipment. Fischer-Tropsch wax and heavy products, especially from slurry and fluid bed processes, may contain contamination (such as catalyst fines) that is not adequately removed by filters provided for that purpose. Contamination can create a serious problem if it is introduced into the hydroprocessing reactor with the feed. The contamination can cause partial or even complete plugging of the flow-paths through the catalyst beds as the contamination accumulates on the surfaces and interstices of the catalyst.

The present inventors have found new methods that are effective at removing contamination, which may include particulates, solidified contaminants, soluble contamination, fouling agents, and/or plugging-precursors from the feed stream to a hydroprocessing reactor when that feed comprises a Fischer-Tropsch derived hydrocarbon stream. The consequences of contamination in the Fischer-Tropsch derived hydrocarbon stream typically include a pressure-drop buildup in the hydroprocessing reactor.

In one embodiment of the present invention, contamination is removed from a Fischer-Tropsch derived hydrocarbon stream using the steps:
a) passing the Fischer-Tropsch derived hydrocarbon stream to an treatment zone;
b) providing an ion exchange medium within the treatment zone;
c) contacting the Fischer-Tropsch derived hydrocarbon stream with the ion exchange medium to remove contamination from the Fischer-Tropsch derived hydrocarbon stream, and
d) removing a purified stream from the treatment zone.

An alternative embodiment comprises the steps of:
a) passing a syngas to a Fischer-Tropsch reactor to produce a Fischer-Tropsch derived hydrocarbon stream;
b) filtering the Fischer-Tropsch derived hydrocarbon stream to produce a filtered hydrocarbon stream;
c) passing the filtered hydrocarbon stream to a treatment zone;
d) providing an ion exchange medium within the treatment zone;
e) contacting the filtered hydrocarbon stream with the ion exchange medium within the treatment zone to remove contamination from the filtered hydrocarbon stream;
f) removing a purified stream from the treatment zone; and
g) passing the purified stream to a hydroprocessing reactor.

Present methods may further include, in addition to the ion exchanging step, an acid treatment step that comprises contacting the filtered hydrocarbon stream with an aqueous acidic stream, a distillation step that includes passing the filtered hydrocarbon stream to at least one distillation step, and a catalytic filtering step that contacts the filtered hydrocarbon stream with an active catalyst.

An advantage of the present methods is that plugging of catalyst beds that otherwise would have been caused by contamination in the conventionally filtered Fischer-Tropsch derived hydrocarbon stream is substantially avoided by passing a purified hydrocarbon stream to the hydroprocessing zone.

### BRIEF DESCRIPTION OF THE DRAWINGS

FIG. 1 is an overview of the present process in which the products of a Fischer-Tropsch reaction are filtered, subjected to an ion exchange process, and then sent on to hydroprocessing;

FIG. 2 shows an exemplary ion exchange mechanism that may be used according to embodiments of the present invention;

FIGS. 3A-B show the structures of exemplary 1:1 and 2:1 clay materials that may be used in embodiments of the present invention.

### DETAILED DESCRIPTION OF THE INVENTION

Embodiments of the present invention are directed to the hydroprocessing of products from a Fischer-Tropsch synthesis reaction. The present inventors have observed under certain conditions a tendency for the catalyst beds in the hydroprocessing reactor to become plugged by either particulate contamination, or by soluble contaminants that precipitate out of solution in the vicinity of or within the catalyst beds, thus impeding the flow of material through the hydroprocessing reactor. The contamination may still be present (meaning the problem still exists) even when the Fischer-Tropsch derived hydrocarbon stream is filtered to remove particulate debris larger than about 0.1 microns.

Though not wishing to be bound by any particular theory, the inventors believe the contamination may be present (at least partly) in the Fischer-Tropsch derived hydrocarbon stream in a soluble form, and the contamination may then precipitate out of solution to form solid particulates after the stream is charged to, for example, a hydroprocessing reactor. Typically, after precipitating, the contamination forms solid plugs in the hydroprocessing reactor. Under certain conditions, the plugging occurs in a central portion of the reactor. The spatial extent of the plugging depends on hydroprocessing conditions and catalyst type, where varying space velocities, for example, can compress or spread the plugging over and/or into different regions of the reactor.

The inventors have discovered that the contamination (which may also be described as a “fouling agent” or “plugging precursor”), in both soluble and particulate forms, may be removed from the conventionally filtered Fischer-Tropsch derived product stream using an ion exchanging positioned upstream of the hydroprocessing zone. Embodiments of the present invention include the installation of an ion exchange zone positioned upstream of a hydroprocessing reaction zone. The ion exchange zone contains an ion exchange medium designed to remove contamination from the filtered Fisher-Tropsch derived hydrocarbon stream. The ion exchange event occurs such that the resulting solid contamination has little opportunity to enter (and hence plug) the pores and flow paths of the hydroprocessing catalyst located downstream from the ion exchanging zone. Conducting the ion exchange event upstream of the hydroprocessing zone is clearly advantageous because then precipitation of contamination does not occur within the pores of the hydroprocessing catalyst, the flow paths through the hydroprocessing beds remain open, and a pressure-drop buildup in the hydroprocessing reactor may be substantially avoided.

Thus, embodiments of the present invention include the installation of an ion exchange zone upstream of the catalytic hydroprocessing zone. The ion exchange zone removes both soluble and insoluble contamination from a filtered Fischer-Tropsch derived hydrocarbon stream. Soluble contamination is forced out of solution before it has the opportunity to solidify within downstream hydroprocessing catalyst beds. In this embodiment, the active filtering catalyst is maintained at conditions at which the contamination precipitates from the solution at a desired rate.

While it is not certain whether the contamination is present in the Fischer-Tropsch derived feed as a soluble species, or as an ultra-fine particulate (meaning probably less than about 0.1 microns in size), it is known that the contamination is not generally removed from that hydroprocessing feedstream by conventional filtering.

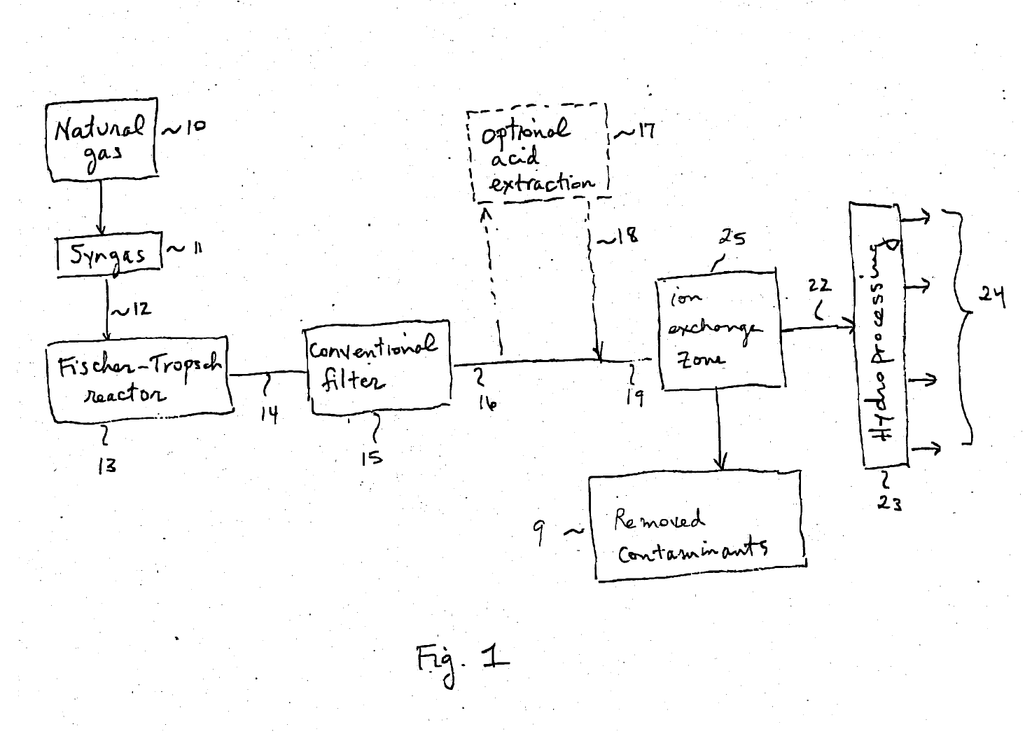

An overview of a process that utilizes an ion exchanging system to purify a Fischer-Tropsch derived hydrocarbon stream is shown in FIG. 1. Referring to FIG. 1, a carbon source such as a natural gas 10 is converted to a synthesis gas 11, which becomes the feed 12 to a Fischer-Tropsch reactor 13. Typically, the synthesis gas 11 comprises hydrogen and carbon monoxide, but may include minor amounts of carbon dioxide and/or water. A Fischer-Tropsch derived hydrocarbon stream 14 may be conventionally filtered in a step 15 to remove particulate contamination greater than about 10 microns in size, and to produce a conventionally filtered hydrocarbon stream 16. The conventionally filtered hydrocarbon stream 16 may then optionally be passed to an acid treatment step 17, in which the filtered hydrocarbon stream 16 is contacted with a dilute aqueous acid to produce an acid treated hydrocarbon stream 18, and a spent acidic aqueous phase (not shown).

Whether or not the optional acid treatment step 17 is carried out, a hydrocarbon feed 19 (which may be either the conventionally filtered product stream 16, or the acid treated stream 18, or combinations thereof) is passed to a ion exchange zone 25, where contamination is removed from the conventionally filtered stream 16, 19 in the presence of an ion exchange medium. Treating the conventionally filtered hydrocarbon stream 16, 19 with the ion exchange medium 25 produces a purified hydrocarbon stream 22 suitable for hydroprocessing. The purified hydrocarbon stream 22 may then be passed to a hydroprocessing zone 23 to provide valuable fuel products 24. The removed (or ion exchanged) contamination is depicted in FIG. 1 as reference numeral 9.

The following disclosure will first focus on the Fischer-Tropsch process itself, and then proceed to a discussion of hydroprocessing reactors and conditions. Then the nature of contamination in general, and the specific problems associated with hydroprocessing catalyst bed plugging will be addressed, before turning to alternative embodiments of the present ion exchange methods.

Fischer-Tropsch Synthesis

A Fischer-Tropsch process may be carried out in the Fischer-Tropsch reactor shown schematically at reference numeral 13 in FIG. 1. The Fischer-Tropsch derived hydrocarbon stream 14 includes a waxy fraction which comprises linear hydrocarbons with a chain length greater than about C₂₀. If the Fischer-Tropsch products are to be used in distillate fuel compositions, they are often further processed to include a suitable quantity of isoparaffins for enhancing the burning characteristics of the fuel (often quantified by cetane number), as well as the cold temperature properties of the fuel (e.g., pour point, cloud point, and cold filter plugging point).

In a Fischer-Tropsch process, liquid and gaseous hydrocarbons are formed by contacting the synthesis gas 11 (sometimes called “syngas”) comprising a mixture of H₂ and CO with a Fischer-Tropsch catalyst under suitable reactive conditions. The Fischer-Tropsch reaction is typically conducted at a temperature ranging from about 300 to 700° F. (149 to 371° C.), where a preferable temperature range is from about 400 to 550° F. (204 to 288° C.); a pressure ranging from about 10 to 600 psia, (0.7 to 41 bars), where a preferable pressure range is from about 30 to 300 psia, (2 to 21 bars); and a catalyst space velocity ranging from about 100 to 10,000 cc/g/hr, where a preferable space velocity ranges from about 300 to 3,000 cc/g/hr.

The Fischer-Tropsch derived hydrocarbon stream 14 may comprise products having carbon numbers ranging from C₁ to C₂₀₀₊, with a majority of the products in the C₅-C₁₀₀ range. A Fischer-Tropsch reaction can be conducted in a variety of reactor types, including fixed bed reactors containing one or more catalyst beds, slurry reactors, fluidized bed reactors, or a combination of these reactor types. Such reaction processes and reactors are well known and documented in the literature.

In one embodiment of the present invention, the Fischer-Tropsch reactor 13 comprises a slurry type reactor. This type of reactor (and process) exhibit enhanced heat and mass transfer properties, and thus is capable of taking advantage of the strongly exothermic characteristics of a Fischer-Tropsch reaction. A slurry reactor produces relatively high molecular weight, paraffinic hydrocarbons when a cobalt catalyst is employed. Operationally, a syngas comprising a mixture of hydrogen (H₂) and carbon monoxide (CO) is bubbled up as a third phase through the slurry in the reactor, and the catalyst (in particulate form) is dispersed and suspended in the liquid. The mole ratio of the hydrogen reactant to the carbon monoxide reactant may range from about 0.5 to 4, but more typically this ratio is within the range of from about 0.7 to 2.75. The slurry liquid comprises not only the reactants for the synthesis, but also the hydrocarbon products of the reaction, and these products are in a liquid state at reaction conditions.

Suitable Fischer-Tropsch catalysts comprise one or more Group VIII catalytic metals such as Fe, Ni, Co, Ru and Re. The catalyst may include a promoter. In some embodiments of the present invention, the Fischer-Tropsch catalyst comprises effective amounts of cobalt and one or more of the elements Re, Ru, Fe, Ti, Ni, Th, Zr, Hf, U, Mg and La on a suitable inorganic support material. In general, the amount of cobalt present in the catalyst is between about 1 and 50 weight percent, based on the total weight of the catalyst composition. Exemplary support materials include refractory metal oxides, such as alumina, silica, magnesia and titania, or mixtures thereof. In one embodiment of the present invention, the support material for a cobalt containing catalyst comprises titania. The catalyst promoter may be a basic oxide such as ThO₂, La₂O₃, MgO, and TiO₂, although promoters may also comprise ZrO₂, noble metals such as Pt, Pd, Ru, Rh, Os, and Ir; coinage metals such as Cu, Ag, and Au; and other transition metals such as Fe, Mn, Ni, and Re.

Useful catalysts and their preparation are known and illustrative, and nonlimiting examples may be found, for example, in U.S. Pat. No. 4,568,663.

Any C₅₊ hydrocarbon stream derived from a Fischer-Tropsch process may be suitably treated using the present process. Typical hydrocarbon streams include a C₅-700° F. stream and a waxy stream boiling above about 550° F., depending on the Fischer-Tropsch reactor configuration. In one embodiment of the present invention, the Fischer-Tropsch derived hydrocarbon stream 14 is recovered directly from the reactor 13 without fractionation. If a fractionation step (not shown in FIG. 1) is performed on the products exiting the Fischer-Tropsch reactor 13, the preferred product of the fractionation step is a bottoms fraction.

Hydroprocessing of the Ion Exchanged Fischer-Tropsch Reaction Products

The product stream 14 from the Fischer-Tropsch reactor 13 may be subjected to a hydroprocessing step. This step may be carried out in the hydroprocessing reactor shown schematically at reference numeral 23 in FIG. 1. The term “hydroprocessing” as used herein refers to any of a number of processes in which the products of the Fischer-Tropsch synthesis reaction produced by reactor 13 are treated with a hydrogen-containing gas; such processes include hydrodewaxing, hydrocracking, hydroisomerization, hydrotreating, and hydrofinishing.

As used herein, the terms “hydroprocessing,” “hydrotreating,” and “hydroisomerization” are given their conventional meaning, and describe processes that are known to those skilled in the art. Hydrotreating refers to a catalytic process, usually carried out in the presence of free hydrogen, in which the primary purpose is olefin saturation and oxygenate removal from the feed to the hydroprocessing reactor. Oxygenates include alcohols, acids. and esters. Additionally, any sulfur which may have been introduced when the hydrocarbon stream was contacted with a sulfided catalyst is also removed.

In general, hydroprocessing reactions may decrease the chain length of the individual hydrocarbon molecules in the feed being hydroprocessed (called “cracking”), and/or increase the isoparaffin content relative to the initial value in the feed (called “isomerization”). In embodiments of the present invention, the hydroprocessing conditions used in the hydroprocessing step 23 produce a product stream 24 that is rich in C₅-C₂₀ hydrocarbons, and with reaction conditions selected to give the desired cold temperature properties (e.g., pour point, cloud point, and cold filter plugging point). Hydroprocessing conditions in zone 23 which tend to form relatively large amounts of C₁₋₄ products are generally not preferred. Conditions which form C₂₀₊ products with a sufficient isoparaffin content to lower the melting point of the wax and/or heavy fraction (such that the particulates larger than 10 microns are more easily removed via conventional filtration) are also preferred.

In some embodiments of the present invention, it may be desirable to keep the amount of cracking of the larger hydrocarbon molecules to a minimum, and in these embodiments a goal of the hydroprocessing step 23 is the conversion of unsaturated hydrocarbons to either fully or partially hydrogenated forms. A further goal of the hydroprocessing step 23 in these embodiments is to increase in the isoparaffin content of the stream relative to the starting value of the feed.

The hydroprocessed product stream 24 may optionally be combined with hydrocarbons from other sources such as gas oils, lubricating oil stocks, high pour point polyalphaolefins, foots oil (oil that has been separated from an oil and wax mixture), synthetic waxes such as normal alpha-olefin waxes, slack waxes, de-oiled waxes, and microcrystalline waxes.

Hydroprocessing catalysts are well known in the art. See, for example, U.S. Pats. 4,347,121, 4,810,357, and 6,359,018 for general descriptions of hydroprocessing, hydroisomerization, hydrocracking, hydrotreating, etc., and typical catalysts used in such processes.

Contamination and Hydroprocessing Catalyst Bed Plugging

As noted above, the Fischer-Tropsch derived hydrocarbon stream 14, 16 may cause plugging of catalyst beds in a hydroprocessing reactor due to contaminants, particulate contamination, soluble contamination, fouling agents, and/or plugging precursors present in the stream 14, 16. The terms particulates, particulate contamination, soluble contamination, fouling agents, and plugging precursors will be used interchangeably in the present disclosure, but the phenomenon will in general be referred to as “contamination,” keeping in mind that the entity that eventually plugs the hydroprocessing catalyst bed may be soluble in the feed at some time prior to the plugging event. The plugging event is a result of the contamination (which eventually takes a particulate form), being filtered out of the hydroprocessing feed by the catalyst beds of the hydroprocessing reactor. According to embodiments of the present invention, a catalytic filtering step 20 is used to remove soluble contamination, fouling agents, and plugging precursors from the Fischer-Tropsch derived hydrocarbon stream 14, 16 such that plugging of the catalyst beds of the hydroprocessing reactor 23 is substantially avoided.

It may be beneficial to address contamination in general before discussing the details of the present catalytic filtration process. Contamination of the Fischer-Tropsch paraffinic product stream 14, 16 can originate from a variety of sources, and, in general, methods are known in the art for dealing with at least some of the forms of the contamination. These methods include, for example, separation, isolation, non-catalytic (conventional) filtration, and centrifugation. Inert impurities such as nitrogen and helium can usually be tolerated, and no special treatment is required.

In general, however, the presence of impurities such as mercaptans and other sulfur-containing compounds, halogen, selenium, phosphorus and arsenic contaminants, carbon dioxide, water, and/or non-hydrocarbon acid gases in the natural gas 10 or syngas 11 is undesirable, and for this reason they are preferably removed from the syngas feed before performing a synthesis reaction in the Fischer-Tropsch reactor 13. One method known in the art includes isolating the methane (and/or ethane and heavier hydrocarbons) component in the natural gas 10 in a de-methanizer, and then de-sulfurizing the methane before sending it on to a conventional syngas generator to provide the synthesis gas 11. In an alternative prior art method ZnO guard beds may be used, and may even be the preferred way to remove sulfur impurities.

Particulate contamination is usually addressed by conventional filtering. Particulates such as catalyst fines that are produced in Fischer-Tropsch slurry or fluidized bed reactors may be filtered out with commercially available filtering systems (in an optional filtering step 15) if the particles are larger than about 10 microns in some procedures, and larger than about one micron in others. The particulate content of the Fischer-Tropsch derived hydrocarbon stream 14, 16 (and particularly the waxy fraction thereof) will generally be small, usually less than about 500 ppm on a mass basis, and sometimes less than about 200 ppm on a mass basis. The sizes of the particulates will generally be less than about 500 microns in diameter, and often less than about 250 microns in diameter. In the context of this disclosure, to say that a particle is less than about 500 microns in diameter means that the particle will pass through a screen having a 500 micron mesh size.

The present inventors have found, however, that a significant level of contamination may remain in a Fischer-Tropsch paraffinic product stream even after conventional filtration. Such contamination typically has a high metal content. As previously disclosed, this contamination will usually lead to a plugging problem if left unchecked. A result of the plugging is a decreased hydroprocessing catalyst life.

The contaminants (including metal oxides) that are extracted from the Fischer-Tropsch derived hydrocarbon stream 14, 16, according to embodiments of the present invention, may have both an organic component as well as an inorganic component. The organic component may have an elemental content that includes at least one of the elements carbon, hydrogen, nitrogen, oxygen, and sulfur (C, H, N, O, and S, respectively). The inorganic component may include at least one of the elements aluminum, cobalt, titanium, iron, molybdenum, sodium, zinc, tin, and silicon (Al, Co, Ti, Fe, Mo, Na, Zn, Sn, and Si, respectively).

Ion Exchange Treatment of a Fischer-Tropsch Derived Hydrocarbon Stream

To the inventors' knowledge, ion exchange techniques have not been used heretofore to purify and/or remove contamination from a Fischer-Tropsch waxy product stream. Ion exchange techniques deemed appropriate for use in the present embodiments include the use of ion exchanging polymeric resins and ion exchanging clays.

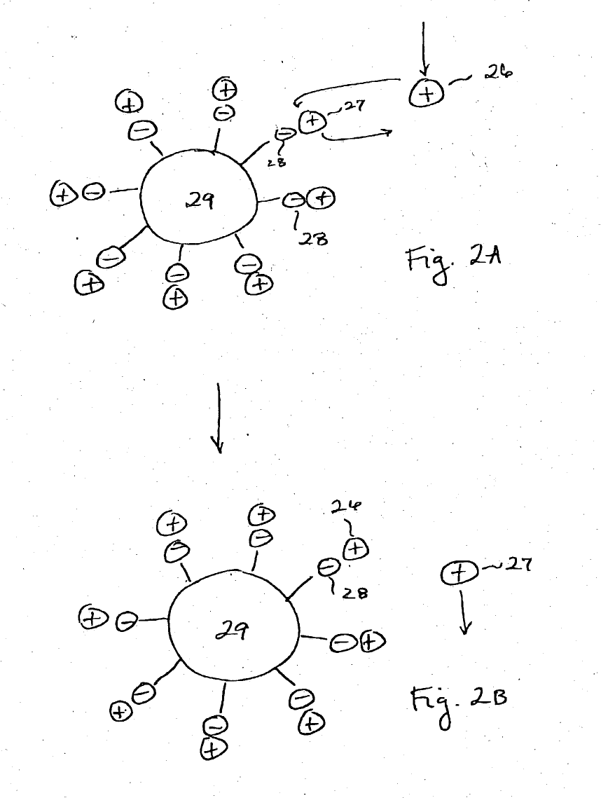

The ion exchange process is illustrated conceptually in FIGS. 2A-B. Referring to FIG. 2A, ion exchange is a process whereby a charged species 26 is removed from the solution in which it is dissolved by competing with an ion of like charge 27 for sites having opposite charge 28 (referred to as attachment sites) located on a resin bead or stationary phase 29. The site of opposite charge 28 may be referred to as a functional group. In the present ion exchange process, it is desirable to remove the charged species 26 from a Fischer-tropsch derived hydrocarbon stream, and this is done by exchanging the charged species 26 with the ion of like charge 27 whose presence in the Fischer-Tropsch derived hydrocarbon stream is either harmless, or more readily removed at a later processing step. In the example depicted in FIGS. 2A-B, the charged species 26 is a positively charged species and the functional group 28 is a negatively charged species, but these charges could be reversed such that the charged species 26 is negatively charged and the functional group 28 is positively charged.

The ion exchange resins 29 suitably employed herein may comprise a water-soluble, copolymeric matrix having attached thereto a plurality of active ion exchange sites 28. The ion exchange sites (or functional groups 28) may be either cation exchange moieties or anion exchange moieties. Cation exchange moieties are generally strong or weak acid groups, such as sulfonic acid, carboxylic acid, phosphoric acid, or like groups. Anion exchange groups are generally strongly or weakly basic groups, such as tertiary amine, quaternary ammonium, phosphonium, sulfonium, and like groups. The resin generally contains a sufficient number of ion exchange groups to impart a concentration of ion exchange sites in the range of from about 0.5 to about 12 meq/gram dry resin and in some cases, from about 4 to about 5.5 meq/gram of dry resin. Cation exchanging sites may be chelating sites which are attracted to or capable of forming coordination complexes with the ion exchanging sites of the contamination to be removed from the Fischer-Tropsch hydrocarbon stream.

The cation exchange resins are capable of exchanging cations. This capability is made possible by functional pendent acid groups on the main polymer chain of the ion exchange resin. Typical functional groups in cation exchange resins are carboxylic acid and/or sulfo groups. The anion exchange resins are those capable of exchanging anions; a capabability provided by ammonium or amine functional pendent groups on the main chain of the resin polymer. Polymers (which may also be referred to as “resins”) having both types of groups are within the purview of embodiments of the present invention. Such resins would be capable of exchanging, and thus removing, both cationic and anionic contamination from a Fischer-Tropsch derived hydrocarbon stream.

The nature of the polymeric background is not particularly critical to the ion exchanging process as long as the resultant polymer is water-insoluble. Accordingly, the polymer may be phenolic, polyethylenic, styrenic, or acrylic polymers, or of any other type which carries or can be made to carry active ion exchange sites. For example, synthetic ion exchange resins such as poly(phenol-formaldehyde), polyacrylic, or polymethacrylic acid or nitrile, amine-epichlorohydrin resins, graft polymers of styrene on polyethylene or polypropylene, poly(2-chloromethyl-1,3-butadiene) and particularly poly(vinylaromatic) resins such as those derived from styrene, alpha-methylstyrene, chlorostyrene, chloromethylstyrene, vinyltoluene, vinylnaphthalene or vinyl pyridine, all of which resins have been suitably crosss-linked to render them insoluble in the solvent media with which they will be contacted and which bear desired ion exchanging sites, are suitable synthetic resins for the polymeric backbone of the ion exchange resins of the present embodiments. Of the known polymerizable monoethylenically unsaturated monomers, the monovinylidene aromatics, such as styrent and monoalkylsubstituted styrenes, such as monovinyltoluene, ethylvinylbenzene and vinyl naphthalene, are exemplary polymers.

The polymers of the present embodiments are crosslinked polymers that may be formed by the addition copolymerization of polymerizable monoethylenically unsaturated monomer or a mixture of such monomer with a crosslinking agent. Typically, the crosslinking agent is a polyethylenically unsaturated monomer such as divinylbenzene. Exemplary cross-linking agents include polyvinylidene aromatics, such as divinylbenzene, divinyltoluene, divinylxylene, divinylnaphthalene, trivinylbenzene, divinyldiphenyl ether, divinyldiphenyl sulphone, and diisopropenelybenzene; ethylene glycol dimethacrylate and divinylsulfide, with the polyvinylidene aromatics, especially divinylbenzene.

The ion exchange resins suitable for use in embodiments of the present invention may be either of the macroporous type or the gel type known in the art. Beads of a gel type of resin, for example, have an available surface area for ion exchange sites that is substantially equal to the outer surface area of the bead. Beads of the macroporous type of resin, however, have an available surface area that includes both the outer surface-of that resin bead, as well as an additional surface area created by minute channels which permeate the structure of the resin bead. The network of channels within the resin bead contribute to, or defines, the bead's porosity. The term “macroporous” is intended to mean that the resin bead has pores or voids present in the resin structure that are large enough to permit entry of the contamination entrained in the Fischer-Tropsch hydrocarbon stream. The so-called “gel-type” resins do not normally contain pores or voids.

According to embodiments of the present invention, the resin beads may take any shape, but are generally either spheroidal, disc-shaped, cylindrical, including hollow cylinders, or generally elongated, such as a fiber. In one embodiment of the present invention, the resin beads are spheroidal, and are about 35-75 microns in diameter.

Suitable polymerizable monoethylenically unsaturated monomers, crosslinking agents, catalysts, polymerization media and methods for preparing the crosslinked addition copolymer in suitable particulate form are well known in the art. Examples of macroporous strong-acid exchange resins are the sulfonated styrene-divinylbenzene copolymers offered commercially under the tradenames AMBERLYST 15, AMBERLYST XN1005, AMBERLYST XN1010, and DOWEX MSC-1. Acid resins of intermediate strength have also been reported, such as those containing functional phosphonic or arsonic groups.

Commercially available cation exchange resins further include DOWEX 50WX2, 50WX4, DOWEX HGR, DOWEX HCR, DOWEX MWC-1, and DOWEX CCR-2.

Macroporous weak-acid resins include those having functional groups that may be phenolic, phosphonous, or carboxylic. Some common weak-acid resins are those derived by crosslinking acrylic, methacrylic, or maleic acid groups by use of a crosslinking agent such as ethylene dimethacrylate or divinylbenzene. DUOLITE C-464 is a tradename applied to a resin having functional carboxylic groups.

Among the macroporous strong-base resins are those which contain quaternary ammonium groups pendent from a poly (styrenedivinylbenzene) matrix AMBERLYST A26, AMBERLYST 27, and DOWEX MSA-1 are tradenames of strong-base resins reportedly having an amine functionality derived from trimethylamine. DOWEX MSA-2 is a tradename of a macroporous strong-base resin reported as having amine functionality derived from dimethylethanolamine.

Macroporous weak-base anion exchange resins generally contain functional groups derived from primary, secondary, or tertiary amines or mixtures of such functionalities. Functional amine groups are derived from condensation products of aliphatic polyamines with formaldehyde or with alkyl dihalides or with epichlorohydrin, such as those under the tradenames DOWEX WGR and DOWEX WGR-2. Other macroporous weak base resins are prepared by reaction of an amine or polyamine with chloromethylated styrene-divinylbenzene copolymer beads, such as DOWEX MWA-1, DOWEX 66, and AMBERLYST A21.

Commercially available anion exchange resins further include DOWEX SBR, DOWEX SBRP, DOWEX 11, DOWEX SAR, and DOWEX MWA-1.

In one embodiment of the present invention, the charged species 26 that is to be removed from the Fischer-Tropsch hydrocarbon stream is an aluminum cation, the ion of like charge 27 is either the H⁺ or Na⁺ cation, and the functional group 28 is an SO₃⁻ ion. The process of sulfonation, which is the the process of introducing SO₃⁻ functional groups into the structure of a synthetic resin, is well known in the art.

According to embodiments of the present invention, clay materials may also be used as the ion exchanging medium which has been positioned within the ion exchange zone 25. As is well known in the art, clay materials may be defined as materials having a particle size less than about 0.002 mm. A clay is a mineral type with specific properties and characteristics. One type of clay useful in the practice of embodiments of the present invention fall into the class of crystalline silicate clays. There are two basic building blocks of these types of clays: the silica tetrahedron and the aluminum octahedron. These building blocks form into sheets which leads to the name “silicate layer clays.” The silicon tetrahedral sheet is defined by sheets of horizontally linked, tetrahedral-shaped units consisting of a centrally four-coordinated silicon atom covalently bonded to four surrounding oxygen atoms arranged in a tetrahedral array. The surrounding oxygen atoms are, in turn, bonded to nearby silicon atoms that serve as inter-unit linkages that hold the sheet together. The octahedral sheet is defined by units of a central, six-coordinated aluminum atom bonded to six surrounding oxygen atoms or hydroxyl ions arranged in an octahedral array.

Clay materials of the present embodiments may undergo isomorphous substitution, whereby an ion in one of the sheets is replaced by another ion of similar size but perhaps different valence. For example, a silicon atom in the silicon tetrahedral sheet may be replaced by an aluminum atom. Stoichiometrically, Si₂O₄ before the substitution becomes SiAlO₄ after the substitution, with the effect that a neutrally charged species becomes a negatively charged species. Likewise, an aluminum atom in the aluminum octahedral sheet may be replaced with a magnesium atom. Stoichiometrically, (OH)₂Al₂O₂ becomes (OH)₂AlMgO₂. Once again, a neutrally charged species becomes a negatively charged species.

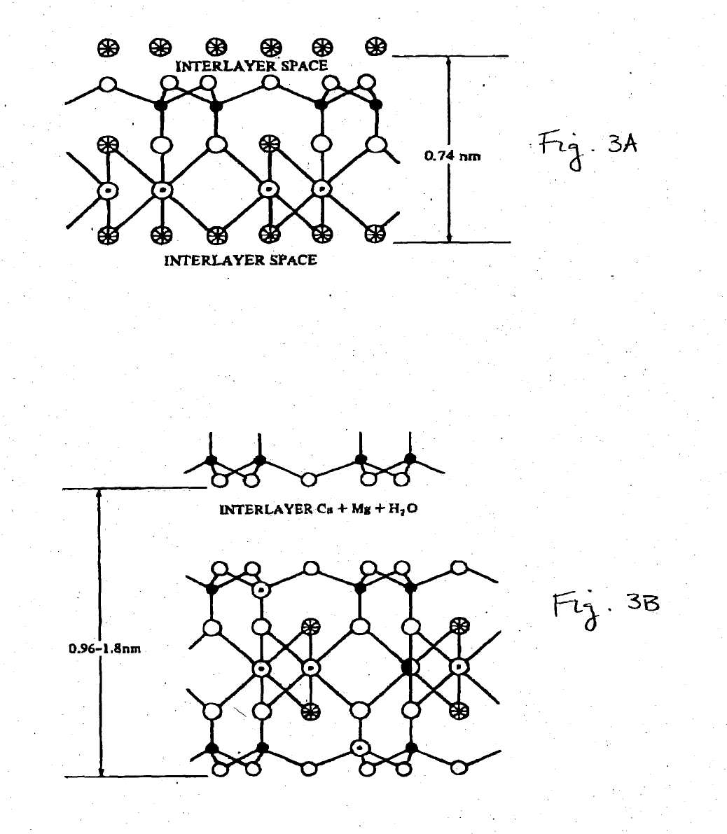

Clay materials are classified according to the numbers and combinations of the constituent structural units, which are the tetrahedral and octahedral sheets, the number of cation substitutions in each sheet (isomorphous substitution), the size and location of a layer charge, and the presence or absence of an interlayer cation. Two general categories of clay materials are defined as being either 1:1 or 2:1. A 1:1 clay consists of one tetradral sheet for each octahedral sheet; a 2:1 clay has two tetrahedral sheets for each octahedral sheet. An exemplary 1:1 clay is kaolinite; exemplary 2:1 clays are the smectites, the micas, and the vermiculites. An example of a mica is illite, and an example of a smectite is montmorillonite. The structure of kaolinite, an exemplary 1:1 clay, and the structure of montmorillonite, an exemplary 2:1 clay, are shown in FIGS. 3A-B, respectively.

Montmorillonite is of particular importance to embodiments of the present invention because of its ability to exchange cations. It has been discussed in an article by P. Bala et al. entitled “Dehydration transformation in Ca-montmorillonite,” Bull. Mater. Sci., Vol. 23, No. 1 (February 2000), pp. 61-67. As discussed above, montmorillonite belongs to the smectite group of clay materials, which have a 2:1 type of layer structure. It is comprised of negatively charged silica sheets held together by charge-balancing counterions such as Mg²⁺, Na⁺, and Ca²⁺. The general chemical formula of montmorillonite is (M_{y}⁺nH₂O)(Al_{4-y}Mg_{y})Si₈O₂₀(OH)₄, where M is the interlayer cation (Mg²⁺, Na⁺, and Ca²⁺). These interlayer cations balance the negative charges which are generated by the isomorphous substitution of Mg²⁺ and Fe²⁺ for Al³⁺ in the octahedral sheets, and Al³⁺ for Si⁴⁺ in the tetrahedral sheets. Besides of the interlayer cations, the interlayer space of the smectite group of clay materials contains large amounts of water molecules, which leads to this material's ability to swell in an aqueous environment.

According to embodiments of the present invention, clay materials may be used to remove contamination from a Fischer-Tropsch derived hydrocarbon stream. In particular, a clay from the smectite group may be used. More particularly, the smectite montmorillonite may be used.

### EXAMPLES

The following examples illustrate various ways in which an ion exchange process may be used to treat a Fischer-Tropsch derived hydrocarbon stream before passing the hydrocarbon stream on to hydroprocessing. The following examples are given for the purpose of illustrating embodiments of the present invention, and should not be construed as being limitations on the scope or spirit of the instant invention.

### Example 1

### Ion Extraction of a Fischer-Tropsch Hydrocarbon Stream Using an Ion Exchange Resin

This example gives the results of an ion exchange process performed on a 5 Fischer-Tropsch derived paraffinic hydrocarbon stream, wherein the process is carried out using a commercially available ion exchange resin. Prior to the acid extraction step, the Fischer-Tropsch product stream was filtered using conventional filtration techniques known to those skilled in the art. The filtered Fischer-Tropsch product stream was then mixed with a commercially available ion exchange resin Dowex HCR-S (Dow 10 Chemical) in a round bottomed flask to which was attached a refluxing condenser. The experiment was carried out with several different ratios of the Fischer-Tropsch derived wax to the ion exchange resin. Each mixture was stirred at 100° C. for one hour, such that the wax was in a molten state.

The levels of contamination in the Fischer-Tropsch wax were compared with the 15 levels in the paraffinic phase measured again after the ion exchange treatment. The elements comprising the contamination, for which an analysis was performed, included aluminum, cobalt, iron, silicon, tin, and zinc. Table 1 shows the amount of the contamination present in the paraffinic phase after the Fischer-Tropsch wax had been treated with the ion exchange resin:

TABLE I

| | Elemental contaminants in the treated | | | | | |
|---|---|---|---|---|---|---|
| | Fischer-Tropsch paraffinic phase (ppm) | | | | | |
| Test mixture | Al | Co | Fe | Si | Sn | Zn |
| Fischer-Tropsch wax | 23 | 2 | 4 | 5 | <1 | <1 |
| (no treatment) | | | | | | |
| 1 part wax: 4 parts water | 25 | 2 | 1 | <1 | 4 | <1 |
| 1 wax: 1 H₂SO₄ (0.1M) | 2 | <1 | 2 | <1 | <1 | <1 |
| 1 part wax: 2 parts simulated | 25 | 2 | 1 | <1 | 5 | <1 |
| reaction water | | | | | | |
| 3 parts wax: 4 parts | 15 | 1 | <1 | 2 | 3 | <1 |
| Dowex HCR-S | | | | | | |
| 3 parts wax: 5 parts | 6 | <1 | <1 | <1 | 1 | <1 |
| Dowex HCR-S | | | | | | |

The numbers in the body of the table represent the amount of an element present in the paraffinic phase after extraction. The technique used to do the elemental analysis was inductively coupled plasma atomic emission spectroscopy (ICP-AES). In this technique, the sample was placed in a quartz vessel (ultrapure grade) to which was added sulfuric acid, and the sample was then ashed in a programmable muffle furnace for 3 days. The ashed sample was then digested with HCl to convert it to an aqueous solution prior to ICP-AES analysis.

The data from Table I clearly show that contaminants are still present in a conventionally filtered Fischer-Tropsch product stream even after that stream had been filtered, but that these contaminants had been substantially removed from the paraffinic stream after it had been extracted with the dilute aqueous acid. The data also shows that the 3 parts wax to 5 parts Dowex HCR-S was more effective at removing contamination than the mixture comprising 3 parts wax to 4 parts Dowex HCR-S.

### Example 2

### Ion Extraction of a Fischer-Tropsch Hydrocarbon Stream Using a Montmorillonite Clay

In this experiment, a montmorillonite clay was used as the ion exchange medium. The results are shown in Table II:

TABLE II

| | Elemental contaminants in the treated | | | | | |
|---|---|---|---|---|---|---|
| | Fischer-Tropsch paraffinic phase (ppm) | | | | | |
| Test mixture | Al | Co | Fe | Si | Sn | Zn |
| Fischer-Tropsch wax | 23 | 2 | 4 | 5 | <1 | <1 |
| (no treatment) | | | | | | |
| 4 parts wax: 1 part | 12.9 | <0.5 | 0.9 | 1.2 | <0.5 | <0.5 |
| Sud-Chemie KP10 | | | | | | |
| 4 parts wax: 1 part | 5.5 | <0.8 | 2.1 | 4.8 | <1.5 | <1 |
| Sud-Chemie K10 | | | | | | |
| 4 parts wax: 1 part | 3.8 | <0.5 | 1.2 | 2.9 | <1.0 | <0.5 |
| T. Actisil | | | | | | |

Table II demonstrates that montmorillonite clays are capable of removing contamination from a Fischer-Tropsch derived wax stream, particularly with regard to the clays Sud-Chemie K10 and T. Actisil.

All of the publications, patents and patent applications cited in this application are herein incorporated by reference in their entirety to the same extent as if the disclosure of each individual publication, patent application or patent was specifically and individually indicated to be incorporated by reference in its entirety.

Many modifications of the exemplary embodiments of the invention disclosed above will readily occur to those skilled in the art. Accordingly, the invention is to be construed as including all structure and methods that fall within the scope of the appended claims.

1. A method of removing contamination from a Fischer-Tropsch derived hydrocarbon stream, the method comprising:
a) passing the Fischer-Tropsch derived hydrocarbon stream to a treatment zone;
b) providing an ion exchange medium within the treatment zone;
c) contacting the Fischer-Tropsch derived hydrocarbon stream with the ion exchange medium within the treatment zone to remove contamination from the Fischer-Tropsch derived hydrocarbon stream, and
d) removing a purified stream from the treatment zone.

2. The method of claim 1, wherein the ion exchange medium in the contacting step comprises a crosslinked, ion exchanging polymeric resin.

3. The method of claim 2, wherein the polymeric resin is a strong-acid exchange resin.

4. The method of claim 2, wherein the polymeric resin is a styrene-divinylbenzene copolymer.

5. The method of claim 2, wherein the ion exchange resin in the contacting step has sulfonium functional groups.

6. The method of claim 1, wherein the ion exchange medium in the contacting step is a clay material.

7. The method of claim 6, wherein the clay material is a 1:1 clay material.

8. The method of claim 7, wherein the 1:1 clay material is kaolinite.

9. The method of claim 6, wherein the clay material is a 2:1 clay material.

10. The method of claim 9, wherein the 2:1 clay material is selected from the group consisting of a a smectite, a mica, and a vermiculite.

11. The method of claim 10, wherein the smectite is montmorillonite.

12. The method of claim 1, wherein the contamination comprises an inorganic component.

13. The method of claim 12, wherein the inorganic component comprises at least one element selected from the group consisting of Al, Co, Ti, Fe, Mo, Na, Zn, Si, and Sn.

14. The method of claim 1, wherein the contamination originates from upstream processing equipment.

15. The method of claim 1, wherein the contamination originates from a catalyst used to produce the Fischer-Tropsch derived hydrocarbon stream.

16. The method of claim 1, wherein the size of the contamination is such that the contamination may be passed through a 1.0 micron filter.

17. The method of claim 1, wherein the contacting step is performed as a batch process.

18. The method of claim 1, wherein the contacting step is performed as a continuous process.

19. The method of claim 1, further including the step of filtering the Fischer-Tropsch derived hydrocarbon stream.

20. The method of claim 1, further including the step of distilling the Fischer-Tropsch derived hydrocarbon stream.

21. The method of claim 1, further including the step of passing the purified stream to a hydroprocessing step.

22. The method of claim 21, wherein the contacting step substantially avoids plugging of catalyst beds in the hydroprocessing reactor.

23. A method of removing contamination from a Fischer-Tropsch derived hydrocarbon stream, the method comprising:
a) passing a syngas to a Fischer-Tropsch reactor to produce a Fischer-Tropsch derived hydrocarbon stream;
b) filtering the Fischer-Tropsch derived hydrocarbon stream to produce a filtered hydrocarbon stream;
c) passing the filtered hydrocarbon stream to a treatment zone;
d) providing an ion exchange medium within the treatment zone;
e) contacting the filtered hydrocarbon stream with the ion exchange medium within the treatment zone to remove contamination from the filtered hydrocarbon stream;
f) removing a purified stream from the treatment zone; and
g) passing the purified stream to a hydroprocessing reactor.
